(19) United States
(12) Patent Application Publication
(10) Pub. No.: US 2019/0326001 A1
(43) Pub. Date: Oct. 24, 2019

(54) Method and Device for Generating Clinical Research Report

(71) Applicant: BEIJING DEEP INTELLIGENT PHARMA CO., LTD., Beijing (CN)

(72) Inventors: Ling Yu, Beijing (CN); Xing Li, Beijing (CN)

(73) Assignee: BEIJING DEEP INTELLIGENT PHARMA CO., LTD.

(21) Appl. No.: 16/201,110

(22) Filed: Nov. 27, 2018

(30) Foreign Application Priority Data
Apr. 19, 2018 (CN) 201810354981.8

(51) Int. Cl. G16H 15/00; G06F 16/31

(52) U.S. Cl. CPC G16H 15/00; G06F 16/328

(57) ABSTRACT

A method and device for generating a clinical research report are provided. The method includes: obtaining a target text and an initial template of a target clinical research report, where the target text includes a target title and a body corresponding to the target title, the initial template includes a preset title and a body part to be filled corresponding to the preset title; matching the preset title with the target title, and copying a body corresponding to the matched target title into the body part to be filled corresponding to the preset title in the initial template, to generate a target template of the target clinical research report, in a case that the matching is successful; and generating the target clinical research report based on the target template.

### CROSS REFERENCE TO RELATED APPLICATION

The present application claims the priority to Chinese Patent Application No. 201810354981.8, titled “METHOD AND DEVICE FOR GENERATING CLINICAL RESEARCH REPORT”, filed on Apr. 19, 2018 with the Chinese Patent Office, which is incorporated herein by reference in its entirety.

### FIELD

The present disclosure relates to the field of clinical medicine, and particularly to a method and device for generating a clinical research report.

### BACKGROUND

In the field of clinical medicine, it is required to perform researches for diseases or drugs, in order to confirm treatment methods for diseases and curative effects for drugs. Clinical researches are usually performed on patients, and are organized and implemented by multi-disciplinary professionals in medical service mechanisms.

Before the clinical research, researchers participating in the clinical research generally need to draft a clinical research protocol and a statistical analysis plan (SAP). The clinical research protocol mainly includes: research background, research objective, overall design of the protocol and implementation of the protocol, etc. The SAP mainly includes research endpoints and methods for statistically analyzing the research endpoints, etc. During the clinical research, statistics analysis is performed on clinical data according to the clinical research protocol and the SAP, to obtain a clinical data chart for clinical research.

When the clinical research ends, researchers need to draft a clinical research report based on the clinical research protocol, the SAP and the data chart. It is usually necessary to draft the clinical research report manually in the conventional technology, resulting in manpower consumption and low efficiency.

### SUMMARY

According to the present disclosure, a method and device for generating a clinical research report is provided, to generate the clinical research report automatically.

A method for generating a clinical research report is provided according to embodiments of the present disclosure, which includes: obtaining a target text and an initial template of a target clinical research report, where the target text includes a target title and a body corresponding to the target title, the initial template includes a preset title and a body part to be filled corresponding to the preset title; matching the preset title with the target title, and copying a body corresponding to the matched target title into the body part to be filled corresponding to the preset title in the initial template, to generate a target template of the target clinical research report, in a case that the matching is successful; and generating the target clinical research report based on the target template.

In an embodiment, the matching the preset title with the target title includes: obtaining a preset keyword of the preset title, and obtaining a target keyword of the target title; and determining whether the preset keyword is identical to the target keyword.

In an embodiment, if the preset title is successfully matched with the target title, the method further includes: determining whether a secondary title of the target title is included in a secondary title of the preset title; and copying the secondary title of the target title into the initial template as the secondary title of the preset title, to generate an interim template of the target clinical research report, in a case that the secondary title of the target title is not included in the secondary title of the preset title. In this case, the generating the target template further includes: copying a body corresponding to the secondary title of the target title into a body part to be filled corresponding to the secondary title of the preset title in the interim template.

In an embodiment, the method further includes: obtaining a clinical data chart including a table title and table data. A title of the interim template is referred to as a first title, the first title includes a preset title and a secondary title of the preset title, and the generating the target template further includes: matching the first title with the table title, and copying table data corresponding to the matched table title into a body part to be filled corresponding to the first title in the interim template, in a case that the matching is successful.

In an embodiment, the method further includes: obtaining a clinical data chart including a table title and table data. In this case, before the body corresponding to the matched target title is copied into the body part to be filled corresponding to the preset title in the initial template, the method further includes: matching the target title with the table title, and in a case that the matching is successful, copying the table data corresponding to the matched table title into the body corresponding to the target title in the target text, so that the body corresponding to the target title includes the table data. Before the body corresponding to the secondary title of the target title is copied into the body part to be filled corresponding to the secondary title of the preset title in the interim template, the method further includes: matching the secondary title of the target title with the table title, and in a case that the matching is successful, copying the table data corresponding to the matched table title into the body corresponding to the secondary title of the target title in the target text, so that the body corresponding to the secondary title of the target title includes the table data.

In an embodiment, the generating the target template further includes: generating a text corresponding to the table data based on the table data by using a text generating model acquired by pre-training, where the text represents meaning of the table data; and adding the generated text corresponding to the table data into a body part to be filled corresponding to the table data in the interim template.

In an embodiment, the process of acquiring the text generating model by pre-training includes: training based on table data in a historical clinical research report and a text corresponding to the table data in the historical clinical research report, to acquire the text generating model.

A device for generating a clinical research report is provided in embodiments of the present disclosure, which includes an obtaining unit, a matching unit and a generating unit. The obtaining unit is configured to obtain a target text and an initial template of a target clinical research report, where the target text includes a target title and a body corresponding to the target title, the initial template includes a preset title and a body part to be filled corresponding to the preset title. The matching unit is configured to match the preset title with the target title, and, copy a body corresponding to the matched target title into the body part to be filled corresponding to the preset title in the initial template, to generate a target template of the target clinical research report, in a case that the matching is successful. The generating unit is configured to generate the target clinical research report based on the target template.

In an embodiment, for matching the preset title with the target title, the matching unit is configured to: obtain a preset keyword of the preset title and obtain a target keyword of the target title; and determine whether the preset keyword is identical to the target keyword.

In an embodiment, if the preset title is successfully matched with the target title, the device further includes: a determining unit configured to determine whether a secondary title of the target title is included in a secondary title of the preset title; and copy the secondary title of the target title into the initial template as the secondary title of the preset title, to generate an interim template of the target clinical research report, in a case that the secondary title of the target title is not included in the secondary title of the preset title. In this case, the matching unit is further configured to: copy a body corresponding to the secondary title of the target title into a body part to be filled corresponding to the secondary title of the preset title in the interim template.

In an embodiment, the obtaining unit is further configured to: obtain a clinical data chart including a table title and table data. A title of the interim template is referred to as a first title, the first title includes a preset title and a secondary title of the preset title, and the matching unit is further configured to: match the first title with the table title, and copy the table data corresponding to the matched table title into a body part to be filled corresponding to the first title in the interim template, in a case that the matching is successful.

In an embodiment, the obtaining unit is further configured to obtain a clinical data chart including a table title and table data. Before the body corresponding to the matched target title is copied into the body part to be filled corresponding to the preset title in the initial template, the matching unit is further configured to: match the target title with the table title, and in a case that the matching is successful, copy the table data corresponding to the matched table title into the body corresponding to the target title in the target text, so that the body corresponding to the target title includes the table data. Before the body corresponding to the secondary title of the target title is copied into the body part to be filled corresponding to the secondary title of the preset title in the interim template, the matching unit is further configured to: match the secondary title of the target title with the table title, and in a case that the matching is successful, copy the table data corresponding to the matched table title into the body corresponding to the secondary title of the target title in the target text, so that the text corresponding to the secondary title of the target title includes the table data.

In an embodiment, the matching unit is further configured to: generate a text corresponding to the table data based on the table data by using a text generating model acquired by pre-training, where the text represents meaning of the table data; and add the generated text corresponding to the table data into a body part to be filled corresponding to the table data in the interim template.

In an embodiment, the process of acquiring the text generating model by pre-training includes: training based on table data in a historical clinical research report and a text corresponding to the table data in the historical clinical research report to acquire the text generating model.

Compared with the conventional technology, the embodiments of the present disclosure have the following advantages. According to the embodiments of the present disclosure, a method and device for generating a clinical research report are provided. The method includes: obtaining a target text and an initial template of a target clinical research report, where the target text includes a target title and a body corresponding to the target title, the initial template includes a preset title and a body part to be filled corresponding to the preset title; matching the preset title with the target title, and copying a body corresponding to the matched target title into the body part to be filled corresponding to the preset title in the initial template, to generate a target template of the target clinical research report, in a case that the matching is successful; and generating the target clinical research report based on the target template. It follows that, with the method and device according to the embodiments of the present disclosure, the target clinical research report can be automatically generated by using the initial template of the target clinical research report and the target text, thereby improving efficiency of generating the clinical research report.

### BRIEF DESCRIPTION OF THE DRAWINGS

In order to illustrate the technical solutions in embodiments of the present disclosure or the conventional technology more clearly, drawings required in the description of embodiments or the conventional technology will be briefly described hereinafter. Obviously, drawings in the following descriptions merely describe some of the embodiments of the present disclosure. Based on these drawings, a person skilled in the art may obtain other drawings without any creative labors.

FIG. 1 is a schematic flowchart of a method for generating a clinical research report according to an embodiment of the present disclosure; and

FIG. 2 is a schematic structural diagram of a device for generating a clinical research report according to an embodiment of the present disclosure.

### DETAILED DESCRIPTION OF EMBODIMENTS

In order to help a person skilled in the art to better understand technical solutions in the present disclosure, the technical solutions in the embodiments of the present disclosure will be described clearly and completely in conjunction with the drawings in the embodiments hereinafter. Obviously, the embodiments to be described are only a part rather than all of the embodiments of the present disclosure. Based on these embodiments, those skilled in the art may obtain other embodiments without any creative effort, and the obtained embodiments also fall in the protection scope of the present disclosure.

Various non-restrictive embodiments of the present disclosure will be described in detail hereinafter in conjunction with the drawings.

### Method Embodiments

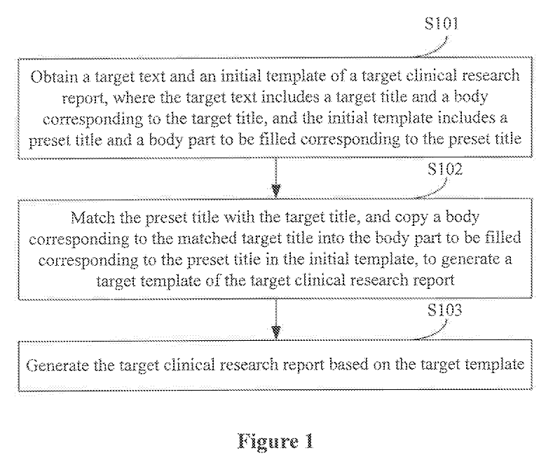

Reference is made to FIG. 1, which is a schematic flowchart of a method for generating a clinical research report according to an embodiment of the present disclosure.

In the embodiment of the present disclosure, for example, the method includes steps S101-S103.

In step S101, a target text and an initial template of a target clinical research report are obtained. The target text includes a target title and a body corresponding to the target title, and the initial template includes a preset title and a body part to be filled corresponding to the preset title.

It should be noted that a clinical research report to be generated is called a target clinical research report in the embodiment of the present disclosure.

It should be noted that the target text is not limited in the embodiment of the present disclosure, and the target text may include a part or all of texts required in generating the target clinical research report.

As an example, the target text may include a clinical research protocol related to the target clinical research report. The clinical research protocol mainly includes research background, research objective, overall design of the protocol and implementation of the protocol, etc.

As another example, the target text may include a statistical analysis plan (SAP) related to the target clinical research report. The SAP mainly includes research endpoints and methods for statistically analyzing the research endpoints, etc.

As another example, the target text may include the above-mentioned clinical research protocol and the above-mentioned SAP.

It should be noted that the target title mentioned in the embodiment of the present disclosure may include a main title, and may further include a secondary title of the main title. The main title may be a first-level title, a second-level title or a third-level title. For example, the target title may include a main title: “1. Research Background”; and the target title may include a secondary title: “1.1 Developments within the Last Five Years”. In another example, the target title may include a main title: “1.1 Developments within the Last Five Years”; and the target title may include a secondary title: “1.1.1 Domestic Developments within the Last Five Years”.

It should be noted that the preset title, similar to the target title, may include a main title and may further include a secondary title of the main title. For the description of the preset title, one may refer to the description of the target title, which is not repeated here.

In step S102, the preset title is matched with the target title; and a body corresponding to the matched target title is copied into a body part to be filled corresponding to the preset title in the initial template to generate a target template of the target clinical research report, in a case that the matching is successful.

It should be noted that successful matching of the preset title with the target title indicates that the meaning of the preset title is identical to the meaning of the target title. Therefore, the body corresponding to the target title in the target text may be copied into the body part to be filled corresponding to the preset title in the initial template, to serve as the body corresponding to the preset title.

It should be noted that the body corresponding to the target title in the target text may be copied into the body part to be filled corresponding to the preset title in the initial template in multiple manners. In a possible manner, the body corresponding to the target title may be copied completely into the body part to be filled corresponding to the preset title. In another possible manner, a part of the body corresponding to the target title may be copied into the body part to be filled corresponding to the preset title. For example, a first preset number of paragraphs contained in the body corresponding to the target title may be copied into the body part to be filled corresponding to the preset title. In another example, a second preset number of sentences contained in the body corresponding to the target title may be copied into the body part to be filled corresponding to the preset title. The first preset number and the second preset number are positive integers, and are not limited in the embodiment.

It should be noted that if the text contained in the target text is an English text, tense transformation can be performed on the body filled in the body part to be filled corresponding to the preset title, after the body corresponding to the target title in the target text is copied into the body part to be filled corresponding to the preset title in the initial template. For example, a simple past tense is changed to a simple future tense, or a simple past tense is changed to a perfect tense.

In step S103, the target clinical research report is generated based on the target template.

It should be understood that the target clinical research report may include a title and a body corresponding to the title. The target template includes a preset title and a body corresponding to the preset title. Therefore, the target clinical research report may be generated based on the target template.

It can be seen that, with the method for generating a clinical research report according to the embodiment of the present disclosure, the target clinical research report is automatically generated by using the initial template of the target clinical research report and the target text, thereby improving efficiency of generating the clinical research report.

In a possible implementation, the process of “matching the preset title with the target title” in step S102 may be achieved by steps A1-B1.

In step A1, a preset keyword of the preset title is obtained, and a target keyword of the target title is obtained.

In step B1, it is determined whether the preset keyword is identical to the target keyword.

It should be understood that the preset title may be not completely identical to the target title since the standard for drafting the target text may be not identical to the standard for drafting the target clinical research report. Therefore, in the embodiments of the present disclosure, a preset keyword of the preset title and a target keyword of the target title may be obtained separately, and the preset keyword is compared with the target keyword. If the preset keyword is identical to the target keyword, it is determined that the preset title is successfully matched with the target title.

It should be noted that the number of the preset keywords of the preset title may be not identical to the number of the target keywords of the target title in practical application. Therefore, in the embodiments of the present disclosure, if a first preset number of preset keywords among the preset keywords of the preset title are included in the target keywords of the target title, it is determined that the preset keywords are identical to the target keywords, and the preset title is successfully matched with the target title.

For example, it may be understood based on Table 1.

TABLE 1

| Preset Title | Target Title |
|---|---|
| 1 INTRODUCTION | 1 INTRODUCTION AND STUDY |
| | RATIONALE |
| 2 STUDY OBJECTIVES | 1.3 Objectives |
| 2.1 Primary Objective | 1.3.1 Primary Objective |
| 2.2 Secondary Objectives | 1.3.2 Secondary Objectives |
| 3 INVESTIGATIONAL PLAN | 1.3.3 Exploratory Objectives |
| | 3 INVESTIGATIONAL PLAN |

As shown in the table, in a case of matching the preset title “1 INTRODUCTION” with the target title “1 INTRODUCTION AND STUDY RATIONALE”, a preset keyword of the preset title “1 INTRODUCTION” is “INTRODUCTION”, and target keywords of the target title include “INTRODUCTION”. “STUDY” and “RATIONALE”. One preset keyword “INTRODUCTION” among the preset keywords of the preset title is included in the target keywords of the target title, so the preset title “1 INTRODUCTION” is successfully matched with the target title “1 INTRODUCTION AN) STUDY RATIONALE”.

Similarly, the preset title “2 STUDY OBJECTIVES” is successfully matched with the target title “1.3 Objectives”; the preset title “2.1 Primary Objective” is successfully matched with the target title “1.3.1 Primary Objective”: the preset title “2.2 Secondary Objectives” is successfully matched with the target title “2.2 Secondary Objectives”; and the preset title “3 INVESTIGATIONAL PLAN” is successfully matched with the target title “3 INVESTIGATIONAL PLAN”.

It should be noted that in practical application, the preset title in the initial template generally includes only first-level titles and second-level titles, and includes no secondary title for the second-level titles. However, titles in the target text are classified in detail, which may include secondary titles that are not included in the preset title. Therefore, secondary titles that are not included in the preset title may be generated based on the target title in the embodiment of the present disclosure. Specifically, if the preset title is successfully matched with the target title, the method further includes: determining whether a secondary title of the target title is included in a secondary title of the preset title: and copying the secondary title of the target title into the initial template as the secondary title of the preset title, to generate an interim template of the target clinical research report, in a case that the secondary title of the target title is not included in the secondary title of the preset title.

It should be noted that when it is determined whether the secondary title of the target title is included in the secondary title of the preset title, the secondary title of the target title is matched with the secondary title of the preset title. If the matching is successful, it is indicated that the secondary title of the target title is included in the secondary title of the preset title. If the matching is unsuccessful, it is indicated that the secondary title of the target title is not included in the secondary title of the preset title.

For example, it may be understood based on Table 1 and Table 2.

TABLE 2

| Preset Title | Target Title |
|---|---|
| 1 INTRODUCTION | 1 INTRODUCTION AND STUDY |
| | RATIONALE |
| 2 STUDY OBJECTIVES | 1.3 Objectives |
| 2.1 Primary Objective | 1.3.1 Primary Objective |
| 2.2 Secondary Objectives | 1.3.2 Secondary Objectives |
| 2.3 Exploratory Objectives | 1.3.3 Exploratory Objectives |
| 3 INVESTIGATIONAL PLAN | 3 INVESTIGATIONAL PLAN |

Since the preset title “2 STUDY OBJECTIVES” is successfully matched with the target title “1.3 Objectives” and the secondary title “1.3.3 Exploratory Objectives” of the target title “1.3 Objectives” is not included in the secondary title of the preset title “2 STUDY OBJECTIVES”, the secondary title “1.3.3 Exploratory Objectives” of the target title “1.3 Objectives” is copied into the initial template as a secondary title “2.3 Exploratory Objectives” of the preset title “2 STUDY OBJECTIVES”.

As mentioned above, the target clinical research report may include a title and a body corresponding to the title. After the secondary title of the preset title is generated, the body corresponding to the secondary title of the preset title may be filled into a body part to be filled corresponding to the secondary title of the preset title.

Namely, the process of generating the target template further includes: copying a body corresponding to the secondary title of the target title into a body part to be filled corresponding to the secondary title of the preset title in the interim template.

It should be noted that after the secondary title of the preset title is generated, the interim template includes the secondary title of the preset title and the body part to be filled corresponding to the secondary title of the preset title.

It should be noted that generally, the target clinical research report may further include table data. Therefore, the table data may be added to the corresponding body part to be filled in the interim template when the target template is to be generated. The table data may be added to the corresponding body part to be filled in the interim template in multiple manners.

In a possible implementation, after the interim template is generated, a clinical data chart including a table title and table data may be obtained. Correspondingly, for the convenience of description, the title of the interim template is referred to as a first title, which includes a preset title and a secondary title of the preset title. When the target template is to be generated, the first title is matched with the table title. In a case that the matching is successful, the table data corresponding to the matched table title is copied into a body part to be filled corresponding to the first title in the interim template.

It should be noted that the clinical data chart is not limited in the embodiment of the present disclosure. As an example, the clinical data chart may be a data chart related to the target text and provided from a statistical part.

It should be noted that the manner for matching the first title with the table title is identical to the manner for matching the preset title with the target title. For the detailed description, one may refer to the description of matching the preset title with the target title as mentioned above, which is not repeated here.

In another possible implementation, after the interim template is generated, a clinical data chart including a table title and table data may be obtained.

Correspondingly, before the process of “copying the body corresponding to the matched target title into the body part to be filled corresponding to the preset title in the initial template” in step S102, the target title may be matched with the table title. In a case that the matching is successful, the table data corresponding to the matched table title is copied into the body corresponding to the target title in the target text, so that the text corresponding to the target title includes the table data.

It should be understood that if the target title is successfully matched with the table title, the table data corresponding to the matched table title is copied into the body corresponding to the target title in the target text, so that the body corresponding to the target title includes the table data. In this way, when the body corresponding to the matched target title is copied into the body part to be filled corresponding to the preset title in the initial template, the table data can be copied into the body part to be filled corresponding to the preset title.

Correspondingly, before the body corresponding to the secondary title of the target title is copied into the body part to be filled corresponding to the secondary title of the preset title in the interim template, the secondary title of the target title may be matched with the table title. In a case that the matching is successful, the table data corresponding to the matched table title is copied into the text corresponding to the secondary title of the target title in the target text, so that the body corresponding to the secondary title of the target title includes the table data.

It should be understood that if the secondary title of the target title is successfully matched with the table title, the table data corresponding to the matched table title is copied into a body corresponding to the secondary title of the target title in the target text, so that the body corresponding to the secondary title of the target title can include the table data. In this way, when the body corresponding to the secondary title of the target title is copied into the body part to be filled corresponding to the secondary title of the preset title in the interim template, the table data can be copied into the body part to be filled corresponding to the secondary title of the preset title.

It should be noted that in practical use, in a case that the target clinical research report includes table data, the table data may be provided with text description for explaining the meaning of the table data, to enhance readability of the target clinical research report.

Therefore, the method for generating a clinical research report provided in the embodiment of the present disclosure may further include steps B1-B2.

In step B1, a text corresponding to the table data is generated based on the table data by using a text generating model acquired by pre-training. The text represents meaning of the table data.

It should be noted that the text generating model is not limited in the embodiment of the present disclosure. In a possible implementation, the text generating model is acquired by training based on table data in a historical clinical research report and a text corresponding to the table data in the historical clinical research report.

It should be noted that the historical clinical research report refers to an existing clinical research report. The historical clinical research report includes table data and a text corresponding to the table data. Therefore, training is performed by using the historical clinical research reports to acquire a mapping relationship between the table data and the text corresponding to the table data, so as to acquire the text generating model. In this way, with the text generating model, the table data is converted into a corresponding text.

It should be understood that the table data may include multiple parameters, which correspond to different clinical research significance. When the training for the text generating model is performed, parameters with greater clinical research significance are determined based on multiple historical clinical research reports, and descriptions of the parameters are arranged in a forward position in the text corresponding to the table data. In this way, when the text corresponding to the table data is generated by using the text generating model, a text corresponding to the parameters with greater clinical research significance is arranged in a forward position in the entire text.

In step B2, the generated text corresponding to the table data is added into a body part to be filled corresponding to the table data in the interim template.

It should be noted that step B2 may be implemented in multiple manners. The manner for implementing step B2 is determined according to practical situations, and is not limited in the embodiment of the present disclosure.

In a possible implementation, the text may be added in front of the table data in the interim template, so that a reader firstly views the meaning of the table data and then views the table data.

In another possible implementation, the text may be added behind the table data in the interim template, so that the reader firstly views the table data and then views the meaning of the table data.

### Device Embodiment

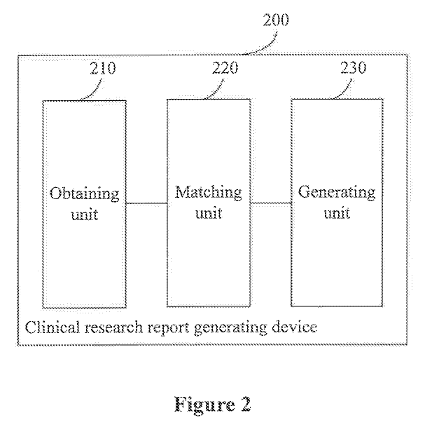

Reference is made to FIG. 2, which is a schematic structural diagram of a device for generating a clinical research report according to an embodiment of the present disclosure.

A device 200 for generating a clinical research report is provided according to an embodiment of the present disclosure. The device 200 may include, for example, an obtaining unit 210, a matching unit 220 and a generating unit 230.

The obtaining unit 210 is configured to obtain a target text and an initial template of a target clinical research report. The target text includes a target title and a body corresponding to the target title, and the initial template includes a preset title and a body part to be filled corresponding to the preset title.

The matching unit 220 is configured to match the preset title with the target title, and copy a body corresponding to the matched target title into the body part to be filled corresponding to the preset title in the initial template to generate a target template of the target clinical research report, in a case that the matching is successful.

The generating unit 230 is configured to generate the target clinical research report based on the target template.

Optionally, for matching the preset title with the target title, the matching unit 220 is configured to: obtain a preset keyword of the preset title and obtain a target keyword of the target title; and determine whether the preset keyword is identical to the target keyword.

Optionally, if the preset title is successfully matched with the target title, the device 200 further includes a determining unit configured to: determine whether a secondary title of the target title is included in a secondary title of the preset title; copy the secondary title of the target title into the initial template as a secondary title of the preset title, to generate an interim template of the target clinical research report, in a case that the secondary title of the target title is not included in the secondary title of the preset title.

In this case, the matching unit 220 is further configured to: copy a text corresponding to the secondary title of the target title into a body part to be filled corresponding to the secondary title of the preset title in the interim template.

Optionally, the obtaining unit 210 is further configured to: obtain a clinical data chart including a table title and table data.

A title of the interim template is referred to as a first title, which includes a preset title and a secondary title of the preset title. The matching unit 220 is further configured to: match the first title with the table title, and copy table data corresponding to the matched table title into a body part to be filled corresponding to the first title in the interim template, in a case that the matching is successful.

Optionally, the obtaining unit 210 is further configured to: obtain a clinical data chart including a table title and table data.

Before the body corresponding to the matched target title is copied into the body part to be filled corresponding to the preset title in the initial template, the matching unit 220 is further configured to: match the target title with the table title, and in a case that the matching is successful, copy the table data corresponding to the matched table title into the body corresponding to the target title in the target text, so that the body corresponding to the target title includes the table data.

Before the body corresponding to the secondary title of the target title is copied into the body part to be filled corresponding to the secondary title of the preset title in the interim template, the matching unit 220 is further configured to: match the secondary title of the target title with the table title, and in a case that the matching is successful, copy the table data corresponding to the matched table title into the body corresponding to the secondary title of the target title in the target text, so that the body corresponding to the secondary title of the target title includes the table data.

Optionally, the matching unit 220 is further configured to: generate a text corresponding to the table data based on the table data by using a text generating model acquired by pre-training, where the text represents meaning of the table data; and add the generated text corresponding to the table data into a body part to be filled corresponding to the table data in the interim template.

Optionally, the acquiring of the text generating model by pre-training includes: training based on table data in a historical clinical research report and a text corresponding to the table data in the historical clinical research report, to acquire the text generating model.

It should be noted that since the device 200 for generating a clinical research report corresponds to the method provided in the method embodiments, for the implementation of each unit of the device 200, one may refer to the description in the method embodiments, which is not repeated here.

It can be seen that, with the device for generating a clinical research report provided in the embodiment of the present disclosure, the target clinical research report is automatically generated by using the initial template of the target clinical research report and the target text, thereby improving efficiency of generating the clinical research report.

After considering the specification and implementing the solution as disclosed herein, a person skilled in the art can readily envisage other embodiments of the present disclosure. The present disclosure aims to cover any variation, use or adaptation of the present disclosure, which follows general principles of the present disclosure and includes well-known knowledge or common technical means in the present technical field that is not disclosed in this disclosure. The specification and the embodiments are merely regarded as examples, and the real scope and spirit of the present disclosure are defined by the appended claims.

It should be appreciated that the present disclosure is not limited to the precise structure as described and shown in the drawings above, and various modifications and improvements may be made without departing from the scope of the present disclosure. The scope of the present disclosure is merely defined by the attached claims.

Merely the preferred embodiments of the present disclosure are described above, and are not intended to limit the scope of the present disclosure. Any changes, equivalents, improvements, and the like made without departing from the spirit and principle of the present disclosure shall fall within the protection scope of the present disclosure.

1. A method for generating a clinical research report, comprising:
obtaining a target text and an initial template of a target clinical research report, wherein the target text comprises a target title and a body corresponding to the target title, and the initial template comprises a preset title and a body part to be filled corresponding to the preset title;
matching the preset title with the target title, and copying a body corresponding to the matched target title into the body part to be filled corresponding to the preset title in the initial template, to generate a target template of the target clinical research report, in a case that the matching is successful; and
generating the target clinical research report based on the target template.

2. The method according to claim 1, wherein the matching the preset title with the target title comprises:
obtaining a preset keyword of the preset title, and obtaining a target keyword of the target title; and
determining whether the preset keyword is identical to the target keyword.

3. The method according to claim 1, wherein if the preset title is successfully matched with the target title, the method further comprises:
determining whether a secondary title of the target title is comprised in a secondary title of the preset title; and copying the secondary title of the target title into the initial template as the secondary title of the preset title to generate an interim template of the target clinical research report, in a case that the secondary title of the target title is not comprised in the secondary title of the preset title;
wherein the generating the target template further comprises:
copying a body corresponding to the secondary title of the target title into a body part to be filled corresponding to the secondary title of the preset title in the interim template.

4. The method according to claim 3, further comprising:
obtaining a clinical data chart comprising a table title and table data;
wherein a title of the interim template is referred to as a first title, the first title comprises a preset title and a secondary title of the preset title, and the generating the target template further comprises:
matching the first title with the table title, and copying table data corresponding to the matched table title into a body part to be filled corresponding to the first title in the interim template, in a case that the matching is successful.

5. The method according to claim 3, further comprising:
obtaining a clinical data chart comprising a table title and table data;
wherein before the copying the body corresponding to the matched target title into the body part to be filled corresponding to the preset title in the initial template, the method further comprises:
matching the target title with the table title; and in a case that the matching is successful, copying the table data corresponding to the matched table title into the body corresponding to the target title in the target text, wherein the text corresponding to the target title comprises the table data;
wherein before the copying the body corresponding to the secondary title of the target title into the body part to be filled corresponding to the secondary title of the preset title in the interim template, the method further comprises:
matching the secondary title of the target title with the table title; and in a case that the matching is successful, copying the table data corresponding to the matched table title into the body corresponding to the secondary title of the target title in the target text, wherein the body corresponding to the secondary title of the target title comprises the table data.

6. The method according to claim 4, wherein the generating the target template further comprises:
generating a text corresponding to the table data based on the table data by using a text generating model acquired by pre-training, wherein the text represents meaning of the table data; and
adding the generated text corresponding to the table data into a body part to be filled corresponding to the table data in the interim template.

7. The method according to claim 5, wherein the generating the target template further comprises:
generating a text corresponding to the table data based on the table data by using a text generating model acquired by pre-training, wherein the text represents meaning of the table data; and
adding the generated text corresponding to the table data into a body part to be filled corresponding to the table data in the interim template.

8. The method according to claim 6, wherein acquiring the text generating model by pre-training comprises: training based on table data in a historical clinical research report and a text corresponding to the table data in the historical clinical research report, to acquire the text generating model.

9. A device for generating a clinical research report, comprising:
an obtaining unit configured to obtain a target text and an initial template of a target clinical research report, wherein the target text comprises a target title and a text corresponding to the target title, and the initial template comprises a preset title and a body part to be filled corresponding to the preset title;
a matching unit configured to match the preset title with the target title, and copy a body corresponding to the matched target title into the body part to be filled corresponding to the preset title in the initial template, to generate a target template of the target clinical research report, in a case that the matching is successful; and
a generating unit configured to generate the target clinical research report based on the target template.

10. The device according to claim 9, wherein if the preset title is successfully matched with the target title, the device further comprises:
a determining unit configured to determine whether a secondary title of the target title is comprised in a secondary title of the preset title; and copy the secondary title of the target title into the initial template as the secondary title of the preset title to generate an interim template of the target clinical research report, in a case that the secondary title of the target title is not comprised in the secondary title of the preset title;
wherein the matching unit is further configured to:
copy a body corresponding to the secondary title of the target title into a body part to be filled corresponding to the secondary title of the preset title in the interim template.

11. The device according to claim 10, wherein the obtaining unit is further configured to:
obtain a clinical data chart comprising a table title and table data;
wherein a title of the interim template is referred to as a fast title, the first title comprises a preset title and a secondary title of the preset title, and the matching unit is further configured to:
match the first title with the table title, and copy table data corresponding to the matched table title into a body part to be filled corresponding to the first title in the interim template, in a case that the matching is successful.

12. The device according to claim 10, wherein the obtaining unit is further configured to obtain a clinical data chart comprising a table title and table data;
wherein before the body corresponding to the matched target title is copied into the body part to be filled corresponding to the preset title in the initial template, the matching unit is further configured to:
match the target title with the table title; and in a case that the matching is successful, copy the table data corresponding to the matched table title into the body corresponding to the target title in the target text, wherein the text corresponding to the target title comprises the table data;
and wherein before the body corresponding to the secondary title of the target title is copied into the body part to be filled corresponding to the secondary title of the preset title in the interim template, the matching unit is further configured to:
match the secondary title of the target title with the table title; and in a case that the matching is successful, copy the table data corresponding to the matched table title into the body corresponding to the secondary title of the target title in the target text, wherein the body corresponding to the secondary title of the target title comprises the table data.

13. The device according claim 11, wherein the matching unit is further configured to:
generate a text corresponding to the table data based on the table data by using a text generating model acquired by pre-training, wherein the text represents meaning of the table data; and
add the generated text corresponding to the table data into a body part to be filled corresponding to the table data in the interim template.

14. The device according claim 12, wherein the matching unit is further configured to:
generate a text corresponding to the table data based on the table data by using a text generating model acquired by pre-training, wherein the text represents meaning of the table data; and
add the generated text corresponding to the table data into a body part to be filled corresponding to the table data in the interim template.
